(19) United States
(12) Patent Application Publication
(10) Pub. No.: US 2005/0031561 A1
(43) Pub. Date: Feb. 10, 2005

(54) Long wearing cosmetic compositions

(76) Inventors: Anjali Abhimanyu Patil, Westfield, NJ (US); Joseph Frank Calello, Bridgewater, NJ (US)

Correspondence Address: JULIE BLACKBURN;REVLON CONSUMER PRODUCTS CORPORATION, 237 PARK AVENUE, NEW YORK, NY, 10017, US

(21) Appl. No.: 10/637,459

(22) Filed: Aug. 8, 2003

(51) Int. Cl. A61K 7/021; A61K 7/06; A61K 7/11

(52) U.S. Cl. 424/63; 424/70.12

(57) ABSTRACT

A long wearing cosmetic composition comprising at least one film forming polymer solvated in volatile linear siloxane, at least one lipophilic particulate, and at least one multifunctional ingredient that interacts with one or more of the other ingredients present, with the ingredients present forming a homogeneous composition, and color cosmetic compositions.

### TECHNICAL FIELD

The invention is in the field of cosmetic compositions, preferably long wearing color cosmetic compositions.

### BACKGROUND OF THE INVENTION

Manufacturers of cosmetic products are on an eternal quest to formulate cosmetic compositions that provide better films on keratinous surfaces. The ideal cosmetic film lasts until the consumer wants to remove it by washing with water or using remover compositions. At the same time the film provides a very natural, aesthetic appearance on the keratinous surface without looking fake or “made up”. A suitable cosmetic film should permit the underlying keratinous surface to breathe, retain moisture, and exhibit a superficially attractive appearance that does not look artificial.

Most often, polymers are incorporated into cosmetic compositions to form the cosmetic film. Generally, such polymers contain many repeating units, or monomers, that give the polymer substantive, film forming properties. Such polymers may be natural or synthetic. Natural polymers such as cellulosics, gums, and resins, have been used as film formers in cosmetics for many years. In more recent years, as polymer chemistry has advanced, polymer manufacturers have been able to manufacture a wide variety of synthetic polymers for use in cosmetics. In general, synthetic polymers fall into one of two classes: silicone polymers (based upon silicon and oxygen), or organic polymers comprised of repeating organic moieties, for example, polymers obtained by polymerizing ethylenically unsaturated monomers such as acrylates or alkylenes, optionally with organic moieties such as amides, urethanes, and the like which may be polymeric or non-polymeric. Certain synthetic polymers that contain both siloxane monomers and organic moieities are also known.

The properties and efficacy of film forming polymers in cosmetic compositions is often improved by formulating them with various types of volatile materials. For example, in transfer resistant compositions, the interaction between the volatile solvent, film forming polymer and pigments, provides the transfer resistant properties to the composition. Cosmetic formulators are continually trying to improve the aesthetics of long wearing cosmetic compositions, by combining various film forming polymers with various types of volatile solvents to maximize cosmetic properties.

It has been discovered that certain volatile linear silicones in combination with at least one film forming polymer, at least one lipophilic particulate, and at least one multifunctional ingredient, provide cosmetic compositions with optimum properties.

It is an object of the invention to provide a cosmetic composition with excellent wear and adhesion to keratinous surfaces.

It is another object of the invention to provide a cosmetic composition that provides a suitable finish to the surface to which it is applied.

It is another object of the invention to provide color cosmetic compositions, specifically lipsticks, that are long wearing and/or transfer resistant.

### SUMMARY OF THE INVENTION

The invention is directed to a cosmetic composition comprising at least one film forming polymer solvated in volatile linear siloxane, at least one lipophilic particulate, and at least one multifunctional ingredient that interacts with the film forming polymer, volatile linear siloxane and lipophilic particulate to provide a homogeneous composition.

### DETAILED DESCRIPTION

All percentages mentioned herein are percentages by weight unless otherwise indicated.

I. Film Forming Polymer

The composition of the invention comprises at least one film forming polymer. The polymer must be capable of being solvated in the linear volatile siloxane that is present. The term “solvated” means that the film forming polymer is either solubilized or dispersed in the linear volatile siloxane. The term “film forming” means that the polymer is capable of forming a film, in particular, a substantive film, on the keratinous surface to which it is applied.

The film forming polymer may be present in any amount, so long as it provides a film either alone, or in combination with other ingredients that may be present. Preferably, the film forming polymer is present in the composition in ranges from about 0.01-99%, preferably about 0.05-85%, more preferably about 0.1-70% by weight of the total composition. The composition may be in the form of an emulsion, that is water-in-oil or oil-in-water wherein the film forming polymer present is solvated in the linear volatile siloxane that forms part of the oily phase. In this case the aqueous phase may also contain film forming polymers that are dispersed or solubilized in the water phase such that the composition comprises an emulsion with film forming polymers dispersed or solvated in the aqueous phase and film forming polymers dispersed or solubilized in the volatile linear siloxane that forms the oily phase. If in the emulsion form, the composition may comprise from about 0.1-99%, preferably about 0.5-97%, preferably about 1-95% by weight of the total composition of water, and about 0. 1-99%, preferably about 0.5-97%, more preferably about 1-95% by weight of the total composition of oily phase ingredients. The compositions of the invention may also be in the anhydrous form, in which case the at least one film forming polymer is solvated in volatile linear siloxane with any other ingredients, such as the lipophilic particulate, and multifunctional ingredient, that may be present. The film forming polymer present in the composition may be a liquid, semi-solid, or solid at room temperature.

Suitable film forming polymers that may be used include, but are not limited to, those set forth herein.

A. Film Forming Siloxane Polymers

Suitable film forming polymers include siloxane polymers that comprise mono-, di-, tri-, or quadrifunctional units. The term “siloxane polymer” means that the siloxane is a polymer, or is comprised of repeating Si—O units or “mers”.

The term “resin” when used in conjunction with the term “siloxane polymer” means that the siloxane polymer provides substantive, resinous, film forming properties when applied to skin, and further that the siloxane contains enough cross-linking to provide substantive, film forming properties. The term cross-linking means a moiety where the silicon atom is bonded to two, three, or four oxygen atoms when the moiety is polymerized with another siloxane unit.

The term monofunctional unit means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); oxyalkylene groups which may be repeating (such as ethylene oxide, propylene oxide) and which may be linked to the siloxy group by alkyl groups having one to three carbon atoms; or hydrogen. In the case where The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

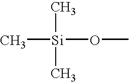

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D”′ designation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula: 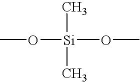
When this difunctional unit is polymerized with one or more of the other units the silicon atom will be bonded to two oxygen atoms, i.e. will share two one-halves of an oxygen atom.

The term “trifunctional siloxy unit” is generally designated by the letter “T” in standard silicone nomenclature. A “T” unit has the general formula:
R₁SiO_{3/2}
wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula: 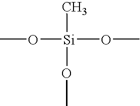
When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

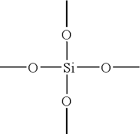

In one preferred embodiment, the film forming silicone polymer that may be used in the compositions of the invention is a siloxane resin, more preferably, comprised of T units and/or Q units either alone or in combination with M units. In addition, there may be one or more of the other types of units present in the polymer, but the film forming siloxane resin polymer is comprised substantially of T and/or Q units, and optionally M units.

Typically T or MT silicones are referred to as silsesquioxanes, and in the case where M units are present methylsilsesquioxanes. Preferred are T silicones having the following general formula:
(R₁SiO_{3/2})x
where x ranges from about 1 to 100,000, preferably about 1-50,000, more preferably about 1-10,000, and wherein R₁ is as defined above.

In another embodiment the preferred silicone resin is an MT resin referred to as polymethylsilsesquioxane which are silsesquioxanes containing methyl groups.

One of the preferred silicone polymers is a polysilsesquioxane manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups, which may be present due to the manner in which such resins are manufactured.

The film forming siloxane polymeric resins that may be used in the composition are made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following: 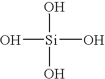
The above hydroxy substituted silane is then condensed or polymerized with other types of silanol substituted units such as: 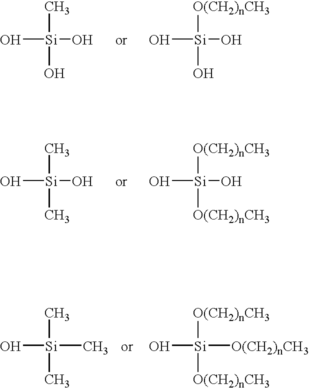
wherein n is 0-10, preferably 0-4.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone film forming polymers that may be used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

In another embodiment of the invention, the film forming siloxane polymer may be one or more linear, high molecular weight silicones that are semi-solids, solids, or gums at room temperature. Such silicones generally comprise M and D units, which may be substituted with substituents other than methyl as noted above. Examples of such silicones include dimethicones having viscosities ranging from about 100,000 to 10 million, or 500,000 to 10 million centipoise at room temperature (25° C.) or dimethicone copolyols having the same viscosity range, the latter being copolymers of monofunctional units, difunctional units, and difunctional units substituted with one or more oxyalkylene groups.

Also suitable are silicone esters as disclosed in U.S. Pat. No. 4,725,658 and U.S. Pat. No. 5,334,737, which are hereby incorporated by reference. Such silicone esters comprise units of the general formula RₐR^{E}_{b}SiO_{[4-(a+b)/2 ]} or R¹³ₓR^{E}_{y}SiO_{1/2}, wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Such silicones may be liquids or solids at room temperature.

Preferred compositions according to the invention contain one or more of a T, MT, MQ, or Q siloxane resin polymer, which may contain one or more D functional units.

B. Copolymers of Silicone and Organic Groups

Another type of film forming polymer that may be used in the compositions of the invention comprises copolymers of siloxane groups and organic groups such as ethylenically unsaturated monomers. Such polymers are generally prepared by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula: 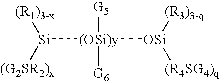
wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z is methylene or propylene.

G₆ is a monovalent moiety which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and

R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.

x is an integer of 0-3;

y is an integer of 5 or greater; preferably 10 to 270, and more preferably 40-270; and

q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved or dispersed in liquids such as isododecane or silicone oils. Preferred is where the polymer is in dry particulate form, and as such it can be solvated in the volatile linear siloxane used in the composition of the invention. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:

A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,

B is at least one reinforcing monomer copolymerizable with A,

C is a monomer having the general formula X(Y)nSi(R)3-m Z.m wherein

X is a vinyl group copolymerizable with the A and B monomers,

Y is a divalent linking group which is alkylene, arylene, alkarylene, and aralkylene of 1 to 30 carbon atoms which may incorporate ester, amide, urethane, or urea groups,

n is zero or 1;

m is an integer of from 1 to 3,

R is hydrogen, C₁₋₄ alkyl, aryl, or alkoxy,

Z is a monovalent siloxane polymeric moiety; and

D is at least one free radically polymerizable acrylate or methacrylate copolymer.

Such polymers and their manufacture are disclosed in U.S. Pat. Nos. 5,209,924 and 4,972,037, which are hereby incorporated by reference. More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula: 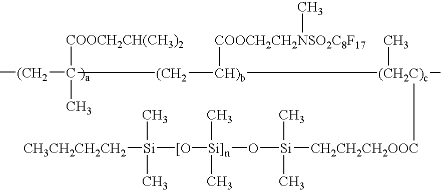
wherein each of a, b, and c has a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least on free radically polymerizable vinyl, methacrylate, or acrylate monomer;

B, when present, is at least one reinforcing monomer copolymerizable with A,

C is a monomer having the general formula:
X(Y)ₙSi(R)₃₋ₘZₘ
wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, C₁₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, ester, or ionic group (such as quaternary ammonium, carboxylate salt or acids such as carboxylic acids, acrylic acids, sulfonic acid or salts, and so on).

The C monomer is as, above defined.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 618,524, issued Apr. 8, 1997, all of which are incorporated by reference herein in their entirety.

C. Polymers from Ethylenically Unsaturated Monomers

Also suitable for use as film forming polymers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

One type of polymer includes one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula:
I. 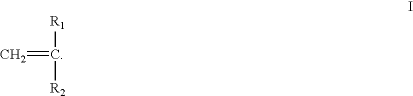
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

Preferably, the monofunctional ethylenically unsaturated monomer is of Formula I, above, wherein R₁ is H or a C₁₋₃₀ alkyl, and R₂ is COOM wherein M is a C₁₋₃₀ straight or branched chain alkyl which may be substituted with one or more hydroxy groups.

More preferably, R₁ is H or CH₃, and R₂ is COOM wherein M is a C₁₋₁₀ straight or branched chain alkyl, which may be substituted with one or more hydroxy groups. In the preferred embodiment of the invention, the monofunctional ethylenically unsaturated monomer is a mixture of monomers of Formula I where in one monomer R₁ is H or CH₃ and R₂ is COOM where M is a C₁₋₁₀ alkyl, and where in the second monomer R₁ is H or CH₃, and R₂ is COOM where M is a C₁₋₁₀ alkyl substituted with one or more hydroxy groups.

Di-, tri- and polyfunctional monomers, as well as oligomers, of the above monofunctional monomers may also be used to form the polymer. Suitable difunctional monomers include those having the general formula: 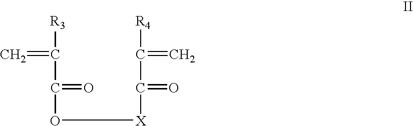
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 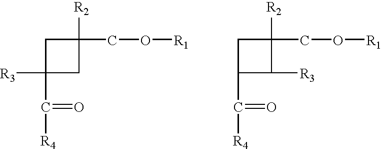
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane.

Another preferred type of organic polymer for use in the compositions of the invention is a suitable film forming polymer for use in the compositions of the invention is a polymer obtained by polymerizing ethylenically unsaturated monomers comprised of vinyl ester groups and optionally organic or silicon groups or other types of ethylenically unsaturated monomers.

Such a film forming polymer comprises vinyl ester monomers having the following general formula: 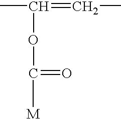
wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The film forming polymer may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula: 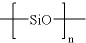
wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

The various organic groups set forth in (D), below, may also be copolymerized with the vinyl ester monomers mentioned herein.

One type of preferred polymer comprises a homopolymer obtained by polymerizing ethylenically unsaturated monomers, all of which contain vinyl ester groups, or a copolymer comprised of vinyl ester monomers in combination with other types of ethylenically unsaturated monomers such as acrylic acid, methacrylic acid or their simple C₁₋₂₂ esters. In the latter case, the polymer preferably comprises more than about 20% by weight of the polymerized vinyl ester monomers, more preferably from about 25 to 99%, even more preferably from about 50-99% of the vinyl ester monomers.

Examples of the most preferred polymers are homopolymers including poly vinyl fatty alkyl esters, polyvinyl short chain alkyl (C₁₋₆) esters, and so on, including but not limited to those set forth herein.

Also suitable are polyvinyl fatty alkyl esters including those where the alkyl chain is a fatty alkyl chain, generally having from 6 to 30 carbon atoms. In the case where the alkyl ester is a fatty alkyl, the fatty alkyl may be a saturated or unsaturated alkyl and the alkyl chain may be branched or straight. Examples of vinyl fatty ester radicals that are suitable include, but are not limited to, vinyl hexanoate, vinyl heptanoate, vinyl octanoate, vinyl nonanoate, vinyl isononanoate, vinyl decanoate, vinyl dodecanoate, vinyl laurate, vinyl stearate, vinyl isostearate, vinyl oleate, vinyl caprylate, vinyl behenate, vinyl palmitate, vinyl myristate, and so on.

One suitable film forming polymer includes polyvinyl neononanoate which may have a mixture of isomers, wherein the monomers are of the general formula: 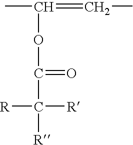
wherein R is H or an alkyl group having 7 carbon atoms, or R+R′+R″=an alkyl group having 7 carbon atoms. This polymer may be purchased from commercial sources including Sigma Aldrich, such polymer having Product No. 56519-94 and the product name polyvinyl neononanoate, mixture of isomers. This polymer may also be purchased from Scientific Polymer Products, under product number 930 having product name poly(vinyl)neononanoate, which is described as a powder material having an approximate molecular weight of about 14,000, and being soluble in the solvents benzene, chloroform, cyclohexane, hexane, MEK (methyl ethyl ketone), methylene chloride, THF, toluene, and xylene.

Another example of a polyvinyl fatty alkyl ester that is suitable for use in the claimed compositions is polyvinyl laurate, a polymer sold by Scientific Polymer Products, Inc. under catalog number 302, having a CAS number 26246-91-3.

Another example of a suitable polymer includes poly(vinyl neodecanoate), which is also sold by Scientific Polymer Products under catalog number 267, and has the CAS number 66987-22-2.

Also suitable are polyvinyl short chain alkyl esters, generally having from one to five carbon atoms. Examples include polyvinyl ethanoate, polyvinyl propionate, polyvinyl butanoate, polyvinyl pentanoate, polyvinyl pivalate, and the like. One example of a polyvinyl short chain alkyl ester is polyvinyl propionate, available from Scientific Polymer Products, Inc., having CAS No. 25035-841.

Another example of a suitable polyvinyl short chain alkyl ester is polyvinyl pivalate, which comprises monomers having the general formula:

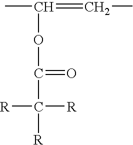

wherein R is methyl.

The preferred film forming polymers used in the compositions of the invention may be in the liquid, solid, or semi-solid form. Preferably the polymers are in the solid form and are solvated in the volatile linear siloxane.

D. Polymers Having Copolymerized Organic Groups

Any of the above mentioned polymers in (A), (B), or (C) above may also be copolymerized with various types of organic groups such as alkylene glycols, including propylene or ethylene glycols, isocyanates, amides, etc.

One type of organic group that can be copolymerized with the above mentioned polymers are urethanes. Urethanes, are generally formed by the reaction of polyhydroxy compounds with diisocyanates, as follows: 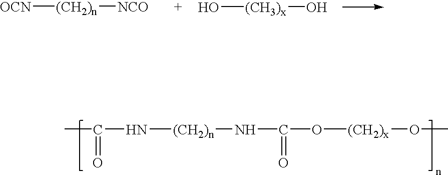
wherein x is 1-1000.

Another type of monomer that may be polymerized with the above comprise amide groups, preferably having the following formula: 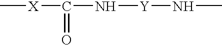
wherein X and Y are each independently linear or branched alkylene having 1-40 carbon atoms, which may be substituted with one or more amide, hydrogen, alkyl, aryl, or halogen substituents.

Another type of organic monomer may be alpha or beta pinenes, or terpenes, abietic acid, and the like.

E. Natural Polymers

Also suitable for use as the film forming polymer are one or more naturally occuring polymeric materials such as resinous plant extracts including such as rosin, shellac, and the like.

II. Volatile Linear Siloxane

The at least one film forming polymer used in the composition of the invention is solvated in volatile linear siloxane. The volatile linear siloxane may be present at ranges from about 0.1 -99%, preferably about 0.5-90%, more preferably about 1-70% by weight of the total composition.

The term “volatile” means that the linear siloxane has a vapor pressure of at least about 1 mm., preferably 2 mm., of mercury at 20° C. The volatile linear siloxane has the general formula: 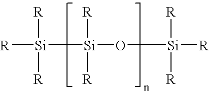
wherein each R is independently butyl, propyl, methyl, ethyl, hydroxyl, or hydrogen, and n is 1 -7, preferably 2-6, more preferably 3-6. Further examples of such linear volatile silicones include hexamethyldisiloxane, octamethyltrisiloxane, decamethyltetrasiloxane, dodecamethylpentasiloxane, tetradecamethylhexasiloxane, and mixtures thereof. In the most preferred embodiment of the invention, the composition comprises only one or more of the volatile linear siloxanes as the total volatile component of the composition.
III. Lipophilic Particulate

The term “lipophilic particulate” means a particulate that is soluble or dispersible in the volatile linear siloxane and/or any other lipophilic ingredients present such as oils, waxes, and the like. The lipophilic particulate may be colored or non-colored (for example, white), and may have a particle size ranging from about 0.005 microns to 500 microns, preferably about 0.005 microns to 200 microns. The lipophilic particulate is preferably present at ranges from about 0.001-95%, preferably about 0.005-90%, more preferably about 0.01-70% by weight of the total composition. Suitable lipophilic particulates include organic and inorganic pigments and particles, or salts thereof

Examples of suitable organic pigments include red, green, blue, yellow, violet, orange, and mixtures thereof. Also suitable are Lakes of such pigments, which means that the organic pigments are reacted with a metal salt such as calcium, aluminum, barium, zirconium, strontium, and the like to form salts. Particularly preferred are aluminum Lakes of the organic pigments, which is where the organic pigment is reacted with aluminum to form the aluminum salt. Formation of the metal salt of the organic pigment will generally also convert the pigment from a water soluble pigment into a water insoluble pigment, and such pigments tend to become even more lipophilic in nature, meaning that they will exhibit appreciable affinity for lipophilic or oily ingredients in the composition. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 CFR 74 101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 1 1, or Lakes thereof.

Also suitable are inorganic pigments that include iron oxides such as red, blue, black, green, and yellow; titanium dioxide, bismuth oxychloride, and the like. Preferred are iron oxides.

Examples of particulate filler-type materials that may be included in the lipophilic particulate include non-pigmentitious particles that generally have a particle size ranging from about 0.002 to 500, preferably 0.5 to 200, microns. Suitable particle fillers include titanated mica, fumed silica, spherical silica, polymethylmethacrylate, micronized teflon, boron nitride, mica, acrylate copolymers, aluminum silicate, aluminum starch octenylsuccinate, bentonite, calcium silicate, cellulose, chalk, polyethylene or other types of spherical beads, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The particulates may also be in the fiber form, such as cellulose fibers, rayon fibers, nylon or silk fibers and the like. Such fibers are generally circular in cross-section and have a discernable length. Preferably the length ranges from 1 to 5 mm.

The above mentioned pigments, powders or fibers may be inherently lipophilic, meaning that they are capable, alone, of being dispersed or solubilized in the oily or lipophilic phase of the composition. For example, certain types of organic pigments may be hydrophilic in nature and will be soluble or dispersible in water, however these pigments may, at the same time be dispersible in an oily phase. Further, in the case of organic pigments, such pigments may be reacted with metal salts to form Lakes, which cause the pigments to exhibit a more lipophilic character. In the compositions of the invention, the pigments may be inherently lipophilic or lipophilic by virtue of surface treatments with lecithin, amino acids, mineral oil, silicone oil or various other agents either alone or in combination, which coat the particulate surface and render the particles more lipophilic in nature. The term “lipophilic” means that the pigment or particles will be solvated, dispersed, and/or compatible with the lipophilic or oily phase of the composition. In the case of an emulsion, a lipophilic particle will have an affinity for the oily phase of the emulsion.

In the most preferred embodiment of the invention the composition comprises organic or inorganic pigments or mixtures thereof in an amount sufficient to provide color to the composition, either alone or in combination with one or more particulate fillers. These particulates are either suspended, dispersed, or solvated in the oily phase of the composition, that phase including the linear volatile siloxane.

IV. The Multifunctional Ingredient

The composition additionally comprises one or more multifunctional ingredients that interact with one or more of the film forming polymer, volatile linear siloxane, and the lipophilic particulate to aid in the formation of a homogeneous composition. The term “multifunctional” means that the ingredient has at least two functional properties, one of which is similar or complementary to one or more of the functional properties found in the film forming polymer, volatile linear siloxane, and the lipophilic particulate. Thus, the similar or complementary property between the multifunctional ingredient and one of the other ingredients in the composition cause the two ingredients to “like” each other in that context, which in turn contributes to formation of a stable and homogeneous composition. For example, the multifunctional ingredient may be an emollient material but at the same time exhibit a small degree of film forming property. In this case the multifunctional ingredient may facilitate the formation of a better film on the keratinous surface than the film that would be obtained with the film forming polymer alone, and at the same time may be compatible with the volatile linear siloxane, thus aiding in solvation of the film forming polymer. In another example, the multifunctional ingredient may have viscosity increasing properties and suspending properties such that it increases viscosity, or thickens, the composition and at the same time aids in the suspension of the particulates found in the composition. Or in yet another example, the multifunctional ingredient may be a volatile solvent such as a volatile hydrocarbon, compatible with the volatile linear siloxane found in the composition, and at the same time have properties that contribute to the solvation of the film forming polymer.

The multifunctional ingredient may be a liquid, solid, or semi-solid. It may be in the form of a polymer, or non-polymeric organic compound or mixture of compounds. Suitable multifunctional ingredients include various types of esters, oils, waxes, and the like. Suggested ranges of the multifunctional ingredient are from about 0.01 -99%, preferably about 0.05-90%, more preferably about 1-80% by weight of the total composition. Suitable multifunctional ingredients include, but are not limited to, those set forth herein.

A. Volatile Cyclic Silicones

Volatile cyclic silicones may be suitable multifunctional ingredients. Such cyclic silicones are of the general formula: 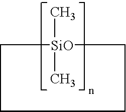
where n=3-6.

Cyclic volatile silicones are available from various commercial sources including Dow Coming Corporation and General Electric. The Dow Coming volatile silicones are sold under the tradenames Dow Coming 244, 245, 344, 345, 246, and 200 fluids. These fluids include octamethylcyclotetrasiloxane, decamethylcyclopentasiloxane, and mixtures thereof.

B. Paraffinic Hydrocarbons

Various straight or branched chain paraffinic hydrocarbons having 5 to 40 carbon atoms, more preferably 8-20 carbon atoms are also suitable as the multifunctional ingredient. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 centipoise at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable. Particularly preferred hydrocarbons, if present, are isohexadecane, isododecane, or mixtures thereof.

C. Nonvolatile Liquids

Also suitable as the multifunctional ingredient are various non-volatile liquid oils such as silicones, esters, and the like. In the most preferred embodiment of the invention, if such non-volatile liquid oils are present, the viscosity may range from about 11 -1000, preferably about 12-800, more preferably about 20-500 at 25° C. Examples of such oils include polyalkylsiloxanes, polyarylsiloxanes, and polyethersiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by references. Further nonlimiting examples of such silicones include dimethicone, phenyl trimethicone, dimethicone copolyol, and so on. Such silicones or organic oils include those further described as follows:
1. Esters

Suitable esters are mono-, di-, and triesters. The composition may comprise one or more esters selected from the group, or mixtures thereof.
(a). Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R-OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on.
(b). Diesters

Suitable diesters are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be subsituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.
(c). Triesters

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.
2. Hydrocarbon Oils

It may be desirable to incorporate one or more non-volatile hydrocarbon oils into the composition for the multifunctional ingredient. The term “nonvolatile” means that the oil has a vapor pressure of less than about 2 mm. of mercury at 20° C.

Suitable nonvolatile hydrocarbon oils include isoparaffins and olefins, preferably those having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, mineral oil, pentahydrosqualene, squalene, squalane, and mixtures thereof.
3. Lanolin Oil

Also suitable for use as the multifunctional ingredient is lanolin oil or derivatives thereof containing hydroxyl, alkyl, or acetyl groups, such as hydroxylated lanolin, isobutylated lanolin oil, acetylated lanolin, acetylated lanolin alcohol, and so on.
4. Glyceryl Esters of Fatty Acids

The multifunctional ingredient may also comprise naturally occuring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.
5. Nonvolatile Silicones

Nonvolatile silicone oils, both water soluble and water insoluble, are also suitable for use as the multifunctional ingredient. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, or polyphenylmethylsiloxane; dimethicone, alkyl substituted dimethicones, and mixtures thereof.

Water soluble, non-film forming silicones such as dimethicone copolyol, dimethiconol, and the like may be used. Such silicones are available from Dow Corning as the 3225C formulation aid, Dow 190 and 193 fluids, or similar products marketed by Goldschmidt under the ABIL tradename.
6. Fluorinated Oils

Also suitable as multifunctional ingredients are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula: 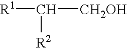
and a fluoroalcohol having the following general formula:
CF₃—(CF₂)ₙ—CH₂—CH₂—OH
wherein n is from 3 to 40.
with a carboxylic acid having the general formula:
R³COOH, or
HOOC—R³—COOH
wherein R¹, R², and R³ are each independently a straight or branched chain alkyl.

The guerbet ester may be a fluoro-guerbet ester, which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyidodecyl meadowfoamate, sold under the tradename Silube GME-F by Siltech, Norcross Georgia.
7. Thixotropic Agents

The multifunctional ingredient may comprise one or more thixotropic agents. The term “thixotropic agent” means an ingredient or combination of ingredients that increase the viscosity of, or thicken, the composition, and which may also suspend the particulates that are found in the composition. In the most preferred embodiment of the invention, the thixotropic agent is a non-matting thixotropic agent, which means that it exhibits a reduced tendency to mute or matte any glossy effect provided by the film forming polymer. Suggested ranges of thixotropic agent range from about 0.01-60%, preferably about 0.05-50%, more preferably about 0.1-45% by weight of the total composition.

One type of non-matting thixotropic agent comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

Also suitable as thixotropic agents are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl

Another type of thixotropic agent that may be used in the composition includes silicas, silicates, silica silylate, and derivatives thereof. These silicas and silicates are generally found in the particulate form. Particularly preferred is silica.

Also suitable thixotropic agents include cross-linked organosiloxane compounds also known as silicone elastomers. Such elastomers are generally prepared by reacting a dimethyl methylhydrogen siloxane with a crosslinking group comprised of a siloxane having an alkylene group having terminal olefinic unsaturation or with an organic group having an alpha or omega unsaturated, e.g. an alpha omega diene. Examples of suitable silicone elastomers for use as thixotropic agents include Dow Coming 9040, sold by Dow Coming, and various elastomeric silicones sold by Shin-Etsu under the KSG tradename including KSG 15, KSG 16, KSG 19 and so on.
8. Natural or Synthetic Waxes

A variety of waxes may be suitable as the multifunctional ingredient, including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, camauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicorie, behenoxy dimethicone, stearyl dimethicone, cetearyl dimethicone, cetyl dimethicone, and so on. Suitable waxes are also set forth in U.S. Pat. No. 5,725,845, which is hereby incorporated by reference in its entirety.
9. Plasticizers

Plasticizers may be suitable multifunctional ingredients. Plasticizers may improve the spreadability and application of the composition to the surface to which it is applied and in some cases will interact with the film forming polymer to make it more flexible. If present, the plasticizer may be found in the oil or water phase if the composition of the invention is in the form of an emulsion, and in the oil or lipophilic phase if the composition is in the anhydrous form. A variety of plasticizers are suitable including Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula: 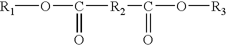
wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl, which may be substituted with one or more hydroxyl groups; and R₃ is a C₃₋₁₀ straight or branched chain alkyl. Examples of such compounds include dioctyl malate, diisopropyl adipate, dibutyl adipate, dibutyl sebacate, dioactyl azelate, dioctyl succinate, dioctyl fumarate, and the like.

In the most preferred embodiment of the invention, the multifunctional ingredient is in the form of a polymer, having repeating monomeric units. For example, a polymeric wax is polyethylene or cetyl dimethicone. The thixotropic agent is an associative polymer, and so on. The multifunctional polymer may be a liquid, solid, or semi-solid, or an oil, wax, thixotropic agent, suspending agent, or the like. It may be a homo-, or copolymer, or a graft or block copolymer.

E. Other Ingredients

The compositions of the invention may comprise a variety of other ingredients such as humectants, preservatives, botanical ingredients, sunscreens, antioxidants, skin care ingredients such as alpha or beta hydroxy acids, emollients, and the like. Examples of such ingredients will be further described herein.
1. Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmits UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Further examples of chemical and physical sunscreens include those set forth below.
(a). UVA Chemical Sunscreens

The term “UVA sunscreen” means a chemical compound that blocks UV radiation in the wavelength range of about 320 to 400 nm. Preferred UVA sunscreens are dibenzoylmethane compounds having the general formula: 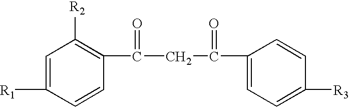
wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isorpoyl-4′-methoxydibenzoymethane, 2-metyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. In one preferred embodiment of the invention the UVA sunscreen is Avobenzone, and it is present at not greater than about 3% by weight of the total composition.
(b). UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. Particularly preferred is Octocrylene, which is 2-ethylhexyl 2-cyano-3,3-diphenylacrylate. Preferred is where the composition contains no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 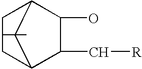
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula: 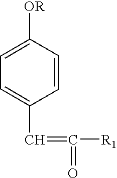
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at nor more than about 3% by weight of the total composition.

Also suitable as the UVB screening agents are various benzophenone derivatives having the general formula: 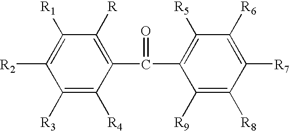
R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula: 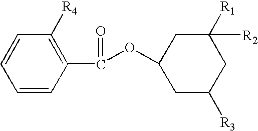
wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula: 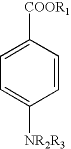
Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 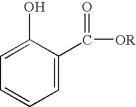
wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.
(c). Physical Sunscreens

The composition may also contain one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-50 microns, preferably less than 1 micron.
2. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenine dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.
3. Humectants

If desired, the compositions of the invention comprise about 0.01-30%, preferably about 0.5-25%, more preferably about 1-20% by weight of the total composition of one or more humectants. Suitable humectants include di- or polyhydric alcohols such as glycols, sugars, and similar materials. Suitable glycols include alkylene glycols such as propylene, ethylene, or butylene glycol; or polymeric alkylene glycols such as polyethylene and polypropylene glycols, including PEG 4-240, which are polyethylene glycols having from 4 to 240 repeating ethylene oxide units. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on.
4. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.
5. Skin Care Actives

It may also be desirable to include one or more skin care actives in the composition. Examples of such skin care actives include alpha or beta hydroxy acids, retinol or fatty acid or salt derivatives thereof (e.g. retinyl palmitate), peptides, and the like. Suitable amounts of skin care actives range from about 0.001-25%, preferably about 0.005-20%, more preferably about 0.01-15% by weight of the total composition.

The invention will be further described in connection with the following examples which are set forth for the purposes of illustration only.

### EXAMPLE 1

| Ingredient | % by Wt. |
|---|---|
| Polyvinyl neononanoate | 34.5 |
| Octamethyltrisiloxane | 37.0 |
| Isododecane/quaternium-18 hectorite | 15.0 |
| propylene carbonate | |
| Isododecane | 3.5 |
| Pigments/Pearls/Mica | 10.0 |

### EXAMPLE 2

| | |
|---|---|
| Octamethyltrisiloxane | 47.50 |
| Polyethylene | 11.50 |
| Ceresin | 8.00 |
| D & C Red #7 Al lake 33% in trioctyldodecyl citrate | 2.00 |
| Red iron oxide 50% in trioctyldodecyl citrate | 9.00 |
| Black iron oxide 50% in trioctyldodecyl citrate | 0.80 |
| TiO2 in trioctyldodecyl citrate | 6.00 |
| Mica | 3.40 |
| Quaternium-18 hectorite 25% in trioctyldodecyl citrate | 2.00 |
| Trioctyldodecyl citrate | 9.30 |
| Methyl paraben | 0.30 |
| Propyl paraben | 0.10 |
| BHT | 0.10 |

### EXAMPLE 3

| | |
|---|---|
| Wacker MK resin | 25.00 |
| Octamethyltrisiloxane | 47.50 |
| Pentaerythrityl tetraoctanoate | 10.00 |
| Cetyl dimethicone | 5.00 |
| Pigments/Pearls/Mica | 10.0 |
| Quaternium-18 hectorite 25% in trioctyldodecyl citrate | 2.00 |
| Methyl paraben | 0.30 |
| Propyl paraben | 0.10 |
| BHT | 0.10 |

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A long wearing cosmetic composition comprising at least one film forming polymer solvated in volatile linear siloxane, at least one lipophilic particulate, and at least one multifunctional ingredient that interacts with one or more of the other ingredients present with the ingredients present forming a homogeneous composition.

2. The composition of claim 1 wherein the volatile linear siloxane is selected from the group consisting of hexamethyldisiloxane, octamethyltrisiloxane, decamethyltetrasiloxane, dodecamethylpentasiloxane, and mixtures thereof.

3. The composition of claim 1 wherein the volatile linear siloxane comprises hexamethyldisiloxane.

4. The composition of claim 1 wherein the volatile linear siloxane comprises octamethyltrisiloxane.

5. The composition of claim 1 wherein the volatile linear siloxane comprises decamethyltetrasiloxane.

6. The composition of claim 1 wherein the volatile linear siloxane comprises dodecamethylpentasiloxane.

7. The composition of claim 1 wherein the film forming polymer is a siloxane polymer, a copolymer of siloxane and one or more ethylenically unsaturated monomers, a siloxane and one or more organic groups, a homo- or copolymer obtained by polymerizing ethylenically unsaturated monomers, a copolymer of ethylenically unsaturated monomers and one or more organic groups.

8. The composition of claim 1 wherein the film forming polymer is a siloxane polymer.

9. The composition of claim 8 wherein the siloxane polymer is a silicone resin.

10. The composition of claim 8 wherein the siloxane polymer is a silicone gum.

11. The composition of claim 1 wherein the film forming polymer comprises a homo- or copolymer of ethylenically unsaturated monomers.

12. The composition of claim 1 comprising two or more film forming polymers.

13. The composition of claim 1 wherein the multifunctional ingredient comprises a polymer.

14. The composition of claim 13 wherein the multifunctional polymer is a silicone oil.

15. The composition of claim 13 wherein the multifunctional polymer is a thixotropic agent.

16. The composition of claim 1 wherein the multifunctional polymer comprises an alkyl dimethicone.

17. The composition of claim 1 wherein the liphophilic particulate comprises iron oxides.

18. The composition of claim 1 wherein the lipophilic particulate comprises organic pigments.

19. The composition of claim 1 wherein the lipophilic particulate comprises a mixture of pigments and non-pigmentitious particulates.

20. The composition of claim 1 wherein the film forming polymer comprises a copolymer of silicone and ethylenically unsaturated monomers, the lipophilic particulate comprises a mixture of pigments and non-pigmentitious powders, and the multifunctional ingredient comprises a polymer.

21. The composition of claim 20 wherein the multifunctional ingredient comprises a silicone oil.

22. The composition of claim 20 wherein the multifunctional ingredient comprises a thixotropic material.

23. The composition of claim 20 wherein the film forming polymer comprises a silicone acrylate copolymer.

24. The composition of claim 20 wherein the liphophilic particulate comprises organic pigments.

25. The composition of claim 21 wherein the liphophilic particulate comprises inorganic pigments.

26. A long wearing cosmetic composition comprising at least one film forming polymer, at least one volatile linear dimethicone, and at least one multifunctional ingredient that is not a polymer.

27. The composition of claim 26 wherein the at least one multifunctional ingredient comprises an ester.

28. The composition of claim 26 wherein the at least one multifunctional ingredient is a wax.

29. The composition of claim 26 wherein the at least one multifunctional ingredient comprises a plasticizer.

30. The composition of claim 1 further comprising a sunscreen.

31. A lipstick composition comprising, by weight of the total composition:
about 0.01-99% of a film forming polymer,
about 0. 1-99% of a volatile silicone selected from the group consisting of octamethyltrisiloxane, decamethyltetrasiloxane, dodecamethylpentasiloxane, tetradecamethylhexasiloxane, and mixtures thereof,
about 0.01-99% of a volatile paraffinic hydrocarbon; and
0.001-95% of a lipophilic particulate selected from the group consisting of inorganic pigment, organic pigment, particulate fillers, and mixtures thereof.

32. The lipstick composition of claim 31 wherein the film forming polymer comprises a silicone.

33. The lipstick composition of claim 31 wherein the film forming polymer comprises a copolymer of silicone and ethylenically unsaturated monomers.

34. The lipstick composition of claim 31 wherein the film forming polymer comprises a polymer made by polymerizing ethylenically unsaturated monomers.

35. The composition of claim 31 wherein the polymer is made by polymerizing one or more silicones, one or more ethylenically unsaturated monomers, and one or more organic groups.

36. The composition of claim 31 wherein the volatile silicone comprises octamethyltrisiloxane.

37. The composition of claim 31 wherein the volatile silicone comprises decamethyltetrasiloxane.

38. The composition of claim 31 wherein the volatile silicone comprises dodecamethylpentasiloxane.

39. The composition of claim 31 wherein the volatile silicone comprises tetradecamethylhexasiloxane.

40. The composition of claim 31 wherein the volatile paraffinic hydrocarbon is selected from the group consisting of isododecane, isohexadecane, and mixtures thereof.
